(19) United States
(12) Patent Application Publication
(10) Pub. No.: US 2024/0201074 A1
(43) Pub. Date: Jun. 20, 2024

(54) DUAL-BEAM OPTOMECHANICAL STEERER AND ASSOCIATED METHODS

(71) Applicant: THE REGENTS OF THE UNIVERSITY OF COLORADO, A BODY CORPORATE, Denver, CO (US)

(72) Inventors: Caroline B. Alden, Boulder, CO (US); Sean C Coburn, Longmont, CO (US); Robert J. Wright, Boulder, CO (US); Gregory B. Rieker, Boulder, CO (US)

(21) Appl. No.: 18/286,101

(22) PCT Filed: Apr. 8, 2022
(86) PCT No.: PCT/US2022/023996

§ 371 Date: Oct. 6, 2023

Related U.S. Application Data
(60) Provisional application No. 63/172,195, filed on Apr. 8, 2021.

(51) Int. Cl. G01N 21/31; G01N 33/00; G02B 26/10

(52) U.S. Cl. CPC G01N 21/31; G01N 33/0027; G02B 26/10; G01N 2201/104

(57) ABSTRACT

A dual-beam optomechanical steerer includes first and second rotators mounted to a two-axis gimbal system. Each rotator is adjustable to control the azimuthal and elevation angles at which an optical transmitter affixed to the rotator transmits a beam of light. Thus, the two-axis gimbal system orients two optical transmitters identically while the first and second rotators orient the two optical transmitters independently with respect to the two-axis gimbal system. Examples of each rotator include a tip-tilt stage, goniometer, and rotation stage. Alternatively, a deflector may be used instead of each rotator. Examples of the deflector include an acousto-optic deflector, translatable lens, and Risley prism. The dual-beam steerer may be used to perform remote gas detection with two separate optical beams.

### RELATED APPLICATIONS

This application claims priority to U.S. Provisional Patent Application No. 63/172,195, filed Apr. 8, 2021, which is incorporated herein by reference in its entirety.

### STATEMENT REGARDING FEDERALLY SPONSORED RESEARCH OR DEVELOPMENT

This invention was made with government support under grant number DE-FE0029168 awarded by the U.S. Department of Energy. The government has certain rights in the invention.

### BACKGROUND

The burning of natural gas emits fewer carbon emissions than the burning of coal, and thus a transition from coal to natural gas may help reduce or revert climate change. The United States is already the world's largest producer of natural gas, outputting over 37 trillion cubic feet in 2018. In the United States, natural gas represents approximately one-third of the nation's entire energy production, the most of any energy type. It is also one of the nation's largest energy sources for electrical generation.

Natural gas is predominantly methane, a potent greenhouse gas. The potency of a greenhouse gas is commonly measured by global warming potential (GWP), which quantifies how much heat the gas traps in the atmosphere, relative to carbon dioxide, over a specific time horizon. By definition, the GWP of carbon dioxide is one. The GWP of methane is 86 over 20 years, and 34 over 100 years.

Significant infrastructure has been constructed, both in the United States and abroad, to extract, process, transport, and utilize natural gas. This infrastructure includes wells and rigs for extraction, pipelines and liquid natural gas (LNG) tankers for transportation, liquification and condensation facilities, processing plants for removing impurities and non-methane components, storage tanks, and industrial boilers (e.g., refineries, power stations, chemical plants) that utilize methane as an energy source for generating heat.

Since methane is a gas, it can easily escape into the atmosphere through emission points that form in equipment and components, such as valves, pipes, connectors, pumps, pressure-relief devices, open-ended lines, and sampling connections. Emissions at a typical facility (e.g., refinery or chemical plant) may arise, for example, from seals and gaskets that are improperly seated or maintained. A typical facility has almost 20,000 valves and connectors, and some facilities may have over 100,000. Failure of any one of these components may result in an emission point. However, emission points may also arise from corrosion of metal components, as well as damage to components due to normal wear and tear and/or anomalous operation.

Therefore, to obtain the full environmental benefit of switching from coal to natural gas, it is important to reduce the number of methane emission points and the quantity of methane emitted by each emission point. The amount of emitted methane (also known as “fugitive emissions”) in the United States is estimated to be between 1.4% and 2.3% of total production per year. Equivalent to 0.5-0.8 trillion cubic feet, these fugitive emissions are enough to heat between 7 and 11 million homes.

In 2016, the United States Environmental Protection Agency (EPA) passed three new rules to help reduce methane emissions in the oil and natural gas industries. These rules include New Source Performance Standards that sets emission limits for methane and requires owners/operators of equipment to find and repair sources of fugitive methane emissions. The EPA estimates that these rules will reduce fugitive methane emissions by 510,000 short tons, or 23 billion cubic feet.

To adhere to the 2016 EPA rules, owners/operators of natural gas well sites, oil well sites, gathering and boosting stations, and compressor stations must survey their equipment for emissions at fixed schedules. Owners/operators must use optical gas imaging (OGI) to conduct these surveys. The most common type of OGI uses an infrared camera that is sensitive between 3.3 and 3.4 μm, where methane has absorption lines. However, the performance of an infrared camera depends on weather conditions (e.g., temperature, wind) as well as the emissivities of materials in the background of the image. As an alternative to OGI, owners/operators may invoke “Method 21” in which surveying is conducted with a portable instrument, such as an organic vapor analyzer.

The 2016 EPA rules also allow the EPA to approve the use of emerging technologies as alternatives to OGI; owners/operators must submit information demonstrating that the alternative technology is capable of achieving methane reductions equivalent to those that can be achieved when OGI or Method 21 is used to find and repair emission points.

In addition to the oil and gas industries, methane emissions are also of concern in agriculture, where global emissions from livestock is estimated at 119 Tg per year (equivalent to 5.9 trillion cubic feet). Other major anthropogenic sources of methane include methane-emitting bacteria that grow in rice paddies (estimated at 115-243 Tg emitted globally per year), biomass burning (estimated at 40-55 Tg emitted globally per year), and landfills (estimated at 40-55 Tg emitted globally per year).

### SUMMARY

The present embodiments include optomechanical beam steerers that can be used to improve the accuracy and data-acquisition speed of remote gas detectors based on optical spectroscopy. Each beam steerer includes a two-axis gimbal system to which two or more rotators are mounted. Affixed to each rotator is an optical transmitter or transceiver that transmits an optical beam (e.g., a laser beam). The two-axis gimbal system moves all of the optical transmitters identically while the rotators are independently controllable to allow each optical transmitter to transmit its optical beam in a unique direction.

The two-axis gimbal system may be thought of as providing “coarse” angular alignment of all the optical transmitters while each rotator provides “fine” angular alignment of the optical transmitter affixed thereto. Thus, in some embodiments, each rotator may have an angular resolution that is finer than that of the two-axis gimbal system. In some embodiments, a beam deflector is used in lieu of a rotator. In this case, each optical transmitter and deflector can be affixed directly to the two-axis gimbal system.

The present embodiment also include methods for ranging using dual-comb spectroscopy. In one of these embodiments, a ranging method includes: transmitting, with an optical transceiver and to a first retroreflector located a first distance from the optical transceiver, a first laser beam comprising an output of a dual frequency comb; receiving, with the optical transceiver, a first reflection of the first laser beam; generating, based on the first reflection, a first interferogram; and determining a first time of a first burst of the first interferogram. The method also includes transmitting, with the optical transceiver and to a second retroreflector located a second distance from the optical transceiver, a second laser beam comprising the output of the dual frequency comb; receiving, with the optical transceiver, a second reflection of the second laser beam; generating, based on the second reflection, a second interferogram; and determining a second time of a second pulse of the second interferogram. The method also includes determining at least one additional piece of data related to the first and second distances and calculating the first and second distances based on the first time, the second time, and the at least one additional piece of data. In some embodiments, the method further includes outputting the first and second distances.

### BRIEF DESCRIPTION OF THE FIGURES

FIG. 1 shows a top view of a laser-based gas detector being used to remotely measure gases within a geographic area, in an embodiment.

FIG. 2 shows a dual-beam optomechanical steerer used with the laser-based gas detector of FIG. 1, in an embodiment.

FIG. 3 shows a dual-beam optomechanical steerer that is similar to the dual-beam optomechanical steerer of FIGS. 1 and 2 except that it includes a first optical deflector and a second optical deflector instead of a first rotator and a second rotators, in an embodiment.

FIGS. 4 and 5 illustrate a method for ranging based on dual comb spectroscopy, in an embodiment.

FIG. 6 is a top view of a geographic region in which an optical transceiver can be rotated to work with seventeen retroreflectors, in an embodiment.

### DETAILED DESCRIPTION

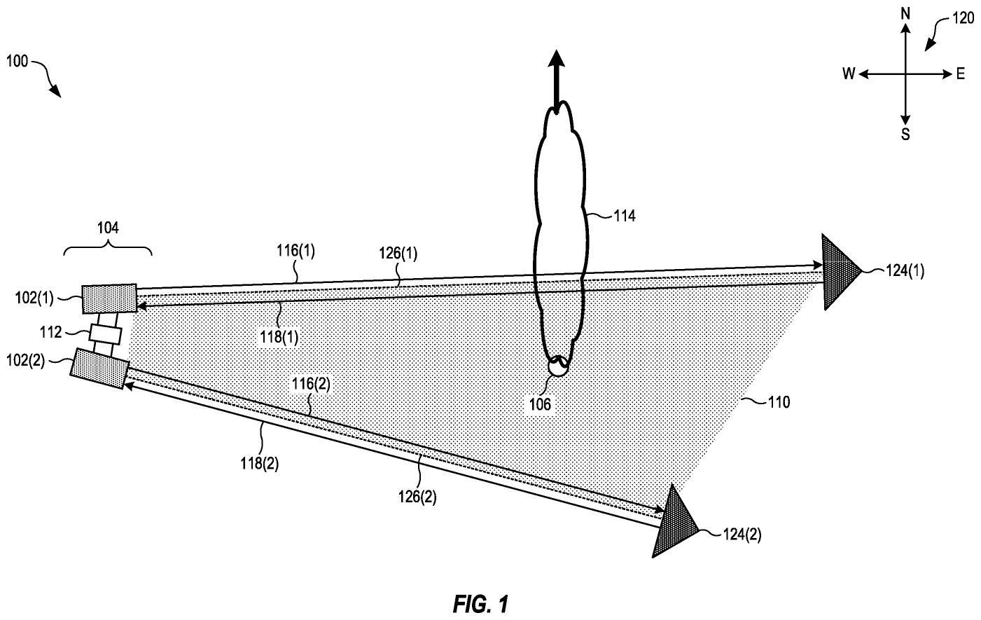

FIG. 1 shows a top view of a laser-based gas detector 100 being used to remotely measure gases within a geographic area 110. The gas detector 100 includes a first optical transceiver 102(1) that transmits a first optical beam 116(1) along a first path 126(1) toward a first retroreflector 124(1) that retroreflects the first optical beam 116(1) into a first retroreflected beam 118(1). The first optical transceiver 102(1) detects the first retroreflected beam 118(1) and the gas detector 100 processes the resulting electronic signal to measure one or more gas species via absorption of the first optical beam 116(1) and first retroreflected beam 118(1). Similarly, the gas detector 100 includes a second optical transceiver 102(2) that transmits a second optical beam 116(2) along a second path 126(2) toward a second retroreflector 124(2) that retroreflects the second optical beam 116(2) into a second retroreflected beam 118(2). The second optical transceiver 102(2) detects the second retroreflected beam 118(2) and the gas detector 100 processes the resulting electronic signal to measure the one or more gas species via absorption of the second optical beam 116(2) and second retroreflected beam 118(2).

The optical transceivers 102(1) and 102(2) are mounted to a dual-beam optomechanical steerer 104 that orients the optical transceivers 102(1) and 102(2). The dual-beam steerer 104 includes a two-axis gimbal system 112 that rotates the optical transceivers 102(1) and 102(2) over an azimuthal range and an elevation angular range. Specifically, the dual-beam steerer 104 orients the first optical transceiver 102(1) such that the first optical beam 116(1) propagates along the first path 126(1) and therefore does not miss the first retroreflector 124(1)). Similarly, the dual-beam steerer 104 orients the second optical transceiver 102(2) such that the second optical beam 116(2) propagates along the second path 126(2) and therefore does not miss the second retroreflector 124(2). As described in more detail below, the dual-beam steerer 104 can orient the transceivers 102(1) and 102(2) independently, even though they are moved identically by the two-axis gimbal system 112.

Although not shown in FIG. 1, each of the optical transceivers 102(1) and 102(2) includes at least one optical source, and associated optics, for generating and transmitting its optical beam 116. The optical source may be a laser, such as a single-frequency laser or a dual frequency comb, typically used for remote gas detection. Alternatively, the optical source may be a fiber-optic cable that receives light from an external laser or light source (i.e., not affixed to the dual-beam steerer 104). Each optical transceiver 102 also includes at least one photodetector, and associated optics, for detecting the corresponding retroreflected beam 118.

In the example of FIG. 1, the gas detector 100 measures emission characteristics of a plume 114 originating at an emission point 106 located in the geographic area 110 bounded by the paths 126(1) and 126(2). Thus, the optical beams 116(1) and 116(2) propagate on opposite sides of the emission point 106. This emission point 106 may be caused by an oil well, pump, storage tank, or other piece of equipment capable of emitting gas. Under different wind conditions (e.g., speed and direction), the plume 114 will flow disproportionately through the paths 126(1) and 126(2). For example, in FIG. 1, where the wind comes from the south (see compass rose 120), the wind pushes the plume 114 northward, and therefore the plume 114 only crosses the first path 126(1) and is only detected by the first optical transceiver 102(1). Accordingly, the first optical beam 116(1) is referred to as the downwind beam while the second optical beam 116(2) is referred to as the upwind beam.

A downwind absorption measurement performed with the downwind beam will show features that are characteristic of one or more species present in both the plume 114 and the background environment. By contrast, an upwind absorption measurement performed with the upwind beam will only show features of the species present in the background. The upwind absorption measurement can be used to correct the downwind absorption measurement (e.g., by performing an inversion) for the presence of the gas species in the background. Therefore, combining the downwind and upwind absorption measurements in this way improves the accuracy with which gas detected by the downwind laser beam can be attributed to originating at the emission point 106. Any disproportionate or differential signature imposed on the downwind and upwind beams by the emitted gas can be used to understand emissions from the emission point 106, and therefore help identify a piece of equipment near or outside the geographic area 110 that could be the source of the emissions.

Examples of gas species that may be measured by the gas detector 100 include, but are not limited to methane, acetylene, carbon dioxide, water vapor, carbon monoxide, hydrogen sulfide, ethylene, ethane, propane, butane, and BTEX (benzene, toluene, ethylbenzene, and xylene). The geographic area 110 may cover several square kilometers, or more, i.e., each path 126 may be several kilometers, or more. For clarity, the dual-beam steerer 104 is not shown to scale in FIG. 1.

Advantageously, the dual-beam steerer 104 orients both of the optical transceivers 102(1) and 102(2) independently, thereby allowing the upwind and downwind absorptions measurements to be performed simultaneously. Specifically, with the dual-beam steerer 104, the optical transceivers 102(1) and 102(2) can simultaneously transmit the optical beams 116(1) and 116(2) along the respective paths 126(1) and 126(2). Similarly, the optical transceivers 102(1) and 102(2) can simultaneously detect the retroreflected beams 118(1) and 118(2) from the respective paths 126(1) and 126(2).

Simultaneous downwind and upwind absorption measurements with two optical transceivers (e.g., the optical transceivers 102(1) and 102(2)) provide several advantages over sequential measurements performed with only one optical transceiver. First, data can be obtained faster since there is no “dead time” between sequential measurements (e.g., the time required to move the gimbal system 112 to reposition the one optical transceiver). With no dead time, more data can be collected over a given period of time, advantageously increasing signal-to-noise ratio, and hence sensitivity. Second, there is no time lag between the downwind measurement and upwind measurement. As a result, there is better rejection of temporal variations in the background concentration since these temporal variations affect both beams simultaneously. This improved background rejection enhances the accuracy of the determined flux. Third, atmospheric modeling is simplified, further enhancing the accuracy of the determined flux.

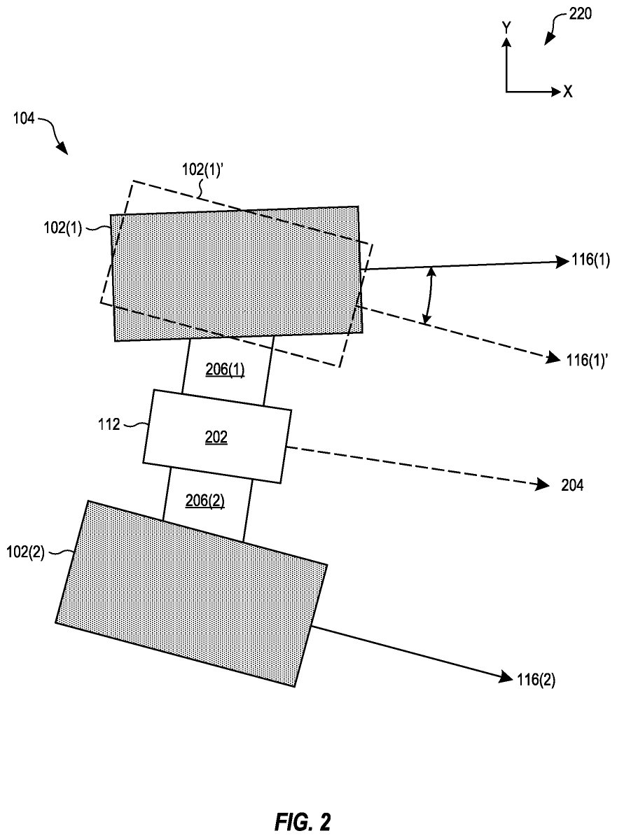

FIG. 2 shows the dual-beam optomechanical steerer 104 of FIG. 1 in more detail. The gimbal system 112 includes two gimbals (not shown) that rotate a center body 202 in both azimuth and elevational angles. The center body 202 defines a global reference direction 204 that changes with the center body 202 as it is rotated. The first optical transceiver 102(1) is mounted to a first rotator 206(1) that, in turn, is mounted to the center body 202. Similarly, the second optical transceiver 102(2) is mounted to a second rotator 206(2) that is also mounted to the center body 202. The rotators 206(1) and 206(2) are rigidly mounted to the center body 202 so that they move identically with the center body 202. The first rotator 206(1) is adjustable to move the first optical transceiver 102(1) to change the propagation direction of the first optical beam 116(1) and therefore the direction from which the first retroreflected beam 118(1) is received. Similarly, the second rotator 206(2) is adjustable to move the second optical transceiver 102(2) to change the propagation direction of the second optical beam 116(2) and therefore the direction from which the second retroreflected beam 118(2) is received.

FIG. 2 shows the first rotator 206(1) rotating the first optical transceiver 102(1) (see rotated transceiver 102(1)′) to change the azimuth of the first optical beam 116(1) (see rotated optical beam 116(1)′). Here, the azimuth is defined in the x-y plane (see right-handed coordinate system 220). Although not shown in FIG. 2, the first rotator 206(1) can additionally or alternatively be controlled to rotate the first optical transceiver 102(1) to change the elevation angle of the first optical beam 116(1). Similarly, the second rotator 206(2) can be controlled to rotate the second optical transceiver 102(2) to change one or both of the azimuth and elevation angle of the second optical beam 116(2). The rotators 206(1) and 206(2) may be independently controllable. Accordingly, the first optical beam 116(1) need not be parallel to the global reference direction 204 or the second optical beam 116(2).

The gimbal system 112 may be rotatable over one or both of a global azimuthal range (e.g., 360°) and a global elevation angular range (e.g., 180°). The gimbal system 112 may provide a common “coarse” angular alignment of the optical transceivers 102(1) and 102(2). The rotators 206(1) and 206(2) may include any mechanism that provides “fine” angular alignment of the optical transceivers 102(1) and 102(2). Thus, in some embodiments, each rotator 206 is, or includes, a mechanism that positions its optical transceiver 102 with a higher angular resolution than the gimbal system 112. Each mechanical rotator 206 may rotate its optical transceiver 102 over an azimuthal range and an elevation angular range. For example, the azimuthal range may be 15° and the elevation angular range may be 5°. The azimuthal and angular ranges may be the same or different. The rotators 206(1) and 206(2) may have the same azimuthal ranges and the second elevation angular ranges.

Examples of mechanisms that may be used for each rotator 206 include a tip-tilt stage, a two-axis goniometer, a ball-and-socket stage, a pitch-and-yaw platform, a one-axis goniometer mounted on a rotation stage, a one-axis tilt stage mounted on a rotation stage, and a pair of stacked wedges independently rotatable about a common axis. Another mechanism may be used without departing from the scope hereof. Each of these mechanisms may be actuated with a motor, piezoelectric actuator (e.g., lead zirconate titanate stack actuator), piezoelectric motor, manual actuator (e.g., micrometer), or another actuator known in the art. A combination of mechanisms may also be used (e.g., a piezoelectric transducer affixed to a manual actuator). In embodiments, the dual-beam steerer 104 includes the actuators and corresponding drive electronics.

In embodiments, the two-axis gimbal system 112 includes a motor. In embodiments, the dual-beam steerer 104 includes the optical transceivers 102(1) and 102(2). In other embodiments, the dual-beam steerer 104 excludes the optical transceivers 102(1) and 102(2). The rotators 206(1) and 206(2) may be configured to receive the respective optical transceivers 102(1) and 102(2). For example, the rotator 206(1) may have mounting holes (e.g., threaded holes), straps, brackets, or other physical means to mount the optical transceiver 102(1) to the rotator 206(1). The rotator 206(2) may be similar configured. In some embodiments, one or both of the optical transceivers 102(1) and 102(2) is an optical transmitter (i.e., not equipped to detect the retroreflected beam 118).

Each of the rotators 206(1) and 206(2) may have a nominal setting. For example, the nominal setting may occur when the rotator 206 is set to the middle of its azimuthal range and the middle of its elevation angular range. In embodiments, the first rotator 206(1) is mounted to the center body 202 so that the first optical transceiver 102(1), when mounted to the first rotator 206(1) in its nominal setting, transmits the first optical beam 116(1) parallel to the global reference direction 204. Similarly, the second rotator 206(2) is mounted to the center body 202 so that the second optical transceiver 102(2), when mounted to the second rotator 206(2) in its nominal setting, transmits the second optical beam 116(2) parallel to the global reference direction 204. In these embodiments, each of the rotators 206(1) and 206(2) may deviate its optical beam 116 from the global reference direction 204 by up to one-half of its second azimuthal range and up to one-half of its second elevation angular range.

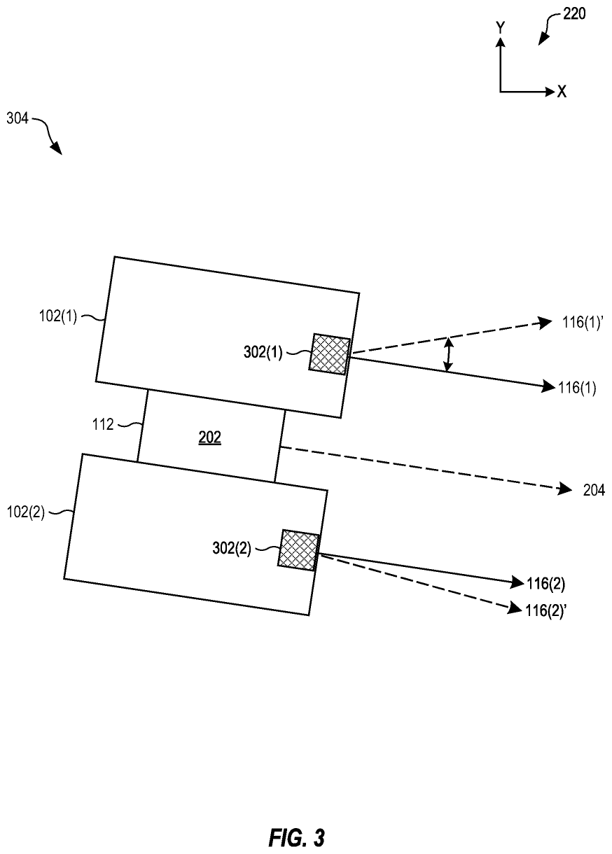

FIG. 3 shows a dual-beam optomechanical steerer 304 that is similar to the dual-beam optomechanical steerer 104 of FIGS. 1 and 2 except that it includes a first optical deflector 302(1) and a second optical deflector 302(2) instead of the first rotator 206(2) and the second rotators 206(2). Specifically, the first optical transceiver 102(1) cooperates with a first optical deflector 302(1) that is operable to deflect the first optical beam 116(1) over an azimuthal range and an elevation angular range. Similarly, the second optical transceiver 302(2) cooperates with a second optical deflector 302(2) that is operable to deflect the second optical beam 116(2) over an azimuthal range and an elevation angular range. The azimuthal ranges of the optical deflectors 302(1) and 302(2) may be the same or different. Similarly, the elevational angular ranges of the optical deflectors 302(1) and 302(2) may be the same or different.

Since the dual-beam steerer 304 excludes the rotators 206(1) and 206(2), each of the optical transceivers 102(1) and 102(2) is rigidly mounted to the center body 202 and therefore moves with the center body 202 as the gimbal system 112 is controlled to change the global reference direction 204. Each of the optical deflectors 302(1) and 302(2) is also rigidly affixed to either the corresponding optical transceiver 102 or the center body 202, and therefore also moves with the center body 202.

Examples of each of the optical deflectors 302(1) and 302(2) include a two-axis translatable lens, a rotatable pair of co-axial refractive optical elements (e.g., a Risley prism pair), a rotatable pair of co-axial diffractive optical elements (e.g. decentered Fresnel lenses), a mirror with adjustable tip and tilt, a two-axis acousto-optic deflector, and a one-axis acousto-optic deflector mounted on an adjustable rotation stage. Another type of optical deflector may be used without departing from the scope hereof. Where the optical deflector 302 uses mechanical actuation, the optical deflector 302 may be actuated with a motor, piezoelectric transducer, piezoelectric motor, manual actuator, or another mechanical actuator known in the art. Alternatively, a combination of mechanical actuators may be used (e.g., a piezoelectric transducer affixed to a manual actuator). In embodiments, the dual-beam steerer 304 includes the mechanical actuators and corresponding drive electronics. In other embodiments, the dual-beam steerer 304 includes electronics for controlling each of the electrically actuated optical deflectors 302(1) and 302(2) (e.g., a radio-frequency signal source and power amplifier for driving an acousto-optic deflector).

Each of the optical deflectors 302(1) and 302(2) may have a nominal setting. For example, the nominal setting may occur when the first optical deflector 302(1) is set to transmit the first optical beam 116(1) without any deviation. In embodiments, the dual-beam steerer 304, when the first optical transceiver 102(1) is mounted to the center body 202, transmits the first optical beam 116(1) parallel to the global reference direction 204 when the first optical deflector 302(1) is at its nominal setting. Similarly, the dual-beam steerer 304, when the second optical transceiver 102(2) is mounted to the center body 202, transmits the second optical beam 116(2) parallel to the global reference direction 204 when the second optical deflector 302(2) is at its nominal setting. In these embodiments, each of the optical deflectors 302(1) and 302(2) deviates its optical beam 116 from the global reference direction 204. Examples of these deviated optical beams are shown in FIG. 3 as optical beams 116(1)′ and 116(2)′. While FIG. 3 shows each of the optical deflectors 302(1) and 302(2) deflecting its optical beam 116 to change its azimuth, each of the optical deflectors 302(1) and 302(2) may additionally or alternatively deflect its optical beam 116 to change the elevation angle.

In some embodiments, one or both of the optical deflectors 302(1) and 302(2) is large enough (in the directions transverse to the optical axis) that an electronic boresight may be placed behind the optical deflector 302 such that a camera of the electronic boresight can view outward through the optical deflector 302 without blocking the optical beam 116. An operator can view the camera output on a screen to visually identify the target retroreflector 124. The operator may manually control one or both of the two-axis gimbal system 112 and optical deflector 302 until the target retroreflector 124 appears on the screen. By placing the electronic boresight behind the optical deflector 302, its field of view will shift as the optical deflector 302 is adjusted.

While the embodiments described above and shown in FIGS. 1-3 have two optical transceivers mounted to one gimbal system, other embodiments have more than two optical transceivers mounted to one gimbal system. For example, in one such embodiment a triple-beam steerer includes three rotators (e.g., the rotators 206(1) and 206(2) in FIG. 2) mounted to the center body of a two-axis gimbal system (e.g., the center body 202 of the two-axis gimbal system 112 in FIG. 2). An optical transceiver (e.g., the optical transceiver 102(1) or 102(2)) may be mounted to each of the three rotators. These three rotators are operable to independently rotate the three optical transceivers. Three deflectors (e.g., the deflectors 302(1) and 302(2) in FIG. 3) may be used instead of three rotators. In another embodiment, a quadruple-beam steerer includes four rotators (or deflectors) mounted to one two-axis gimbal system. In other embodiments, more than four rotators (or deflectors) are mounted to one two-axis gimbal system. In any of the present embodiments, a combination of rotators and deflectors may be used (as opposed to only rotators or only deflectors).

Ranging with DCS

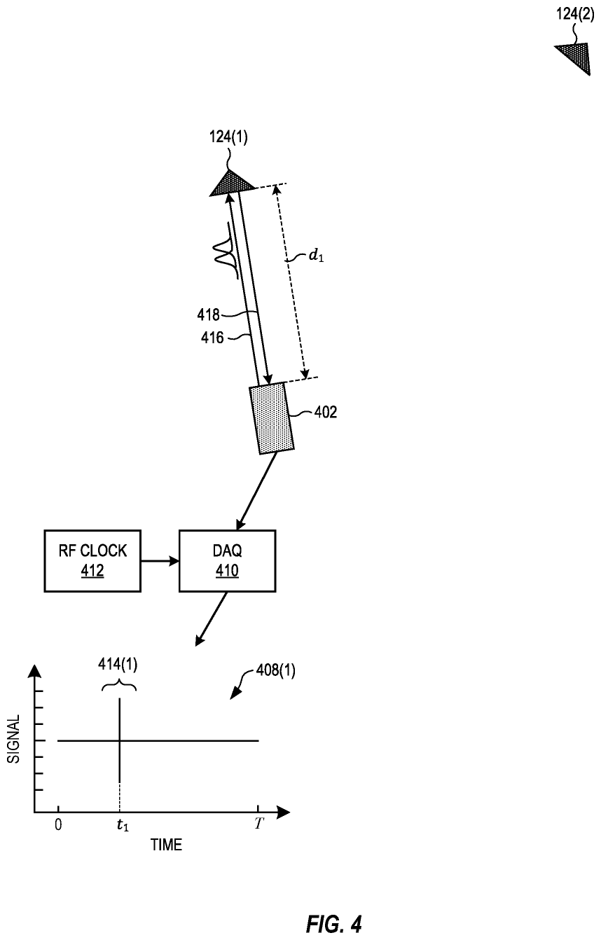

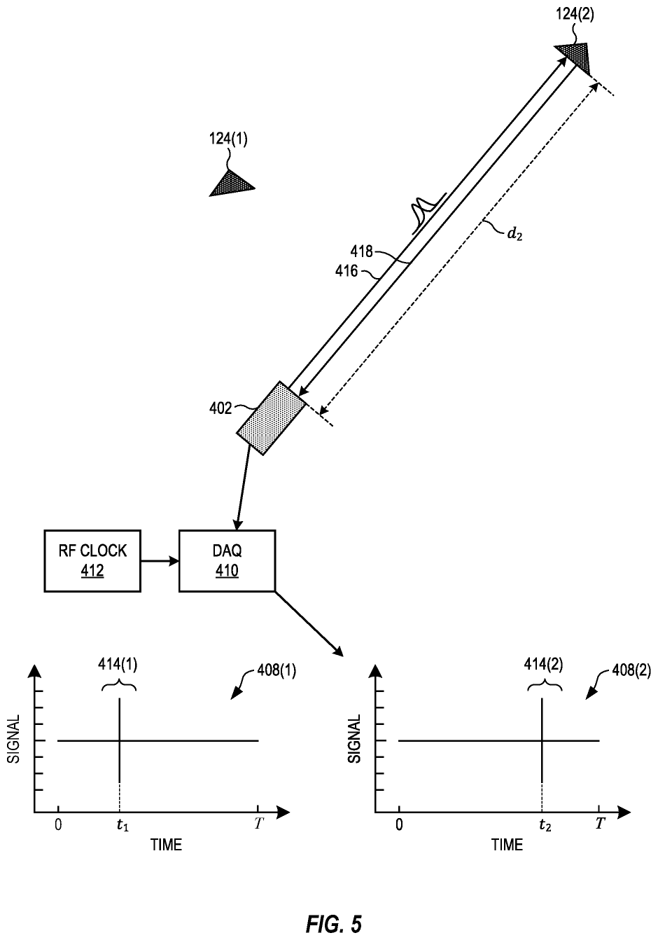

FIGS. 4 and 5 illustrate a method for ranging based on dual comb spectroscopy (DCS). Here, “ranging” means determining a distance between an optical transceiver 402 and each of several retroreflectors 124. The optical transceiver 402 may be one of the optical transceivers 102(1) and 102(2) shown in FIGS. 1-3. Since DCS measurements are integrated over a path, knowledge of the path distance is needed to convert the resulting measurements into concentrations. FIGS. 4 and 5 are best viewed together with the following description.

FIG. 4 shows the optical transceiver 402 transmitting an optical beam 416 toward a first retroreflector 124(1) that retroreflects the optical beam 416 into a retroreflected beam 418. The transceiver 402 detects the retroreflected beam 418, and a data acquisition (DAQ) module 410 processes and digitizes the detected signal to obtain a first interferogram 408(1). The optical beam 416 is an example of the optical beams 116(1) and 116(2) of FIGS. 1-3 and the retroreflected beam 418 is an example of the retroreflected beams 118(1) and 118(2) of FIGS. 1-3. The optical beam 416 is formed a first pulse train that has a first repetition rate fᵣₑₚ⁽¹⁾ and a second pulse train that has a second repetition rate fᵣₑₚ⁽²⁾ that is slightly different from fᵣₑₚ⁽¹⁾(i.e., fᵣₑₚ⁽¹⁾−fᵣₑₚ⁽²⁾≠0). The first interferogram 408(1) exhibits a first burst 414(1) when one pulse from each of the first and second pulse trains overlap upon detection. The interferogram 408(1) has a duration T=1/|fᵣₑₚ⁽¹⁾−fᵣₑₚ⁽²⁾|which corresponds to the next time when one pulse from each of the two pulse trains will again overlap. The center of the first burst 414(1) occurs at a first time t₁ that depends on a first distance d₁ between the transceiver 102 and the first retroreflector 124(1). An RF clock 412 outputs a clock signal at a clock frequency f_{c}, i.e., the points of the interferogram 408(1) spaced by 1/f_{c}.

FIG. 5 shows the optical transceiver 402 transmitting the optical beam 416 toward a second retroreflector 124(2). Again, the optical transceiver 402 detects the resulting retroreflected beam 418 to obtain a second interferogram 408(2). The second interferogram 408(2) has a second burst 414(2) centered at a second time t₂, which depends on a second distance d₂ between the optical transceiver 402 and the second retroreflector 124(2).

The relative difference between the first and second distances is Δd=cΔt/2, where Δt=t₂−t₁ and the factor of 2 accounts for the roundtrip path (i.e., the distance traveled by the optical beam 416 and the distance traveled by the retroreflected beam 418). The factor of 2 should be modified to account for more complex beam-path geometries than shown in FIGS. 4 and 5 (e.g., one-way paths, multiple-leg paths, etc.). In terms of the digitized data of the interferograms 408(1) and 408(2), Δd=cΔn/2f_{c}=c(n₂−n₁)/2f_{c}, where n₂ is the index of the data point of the second interferogram 408(2) corresponding to t₂, and n₁ is the index of the data point of the first interferogram 408(1) corresponding to t₁.

There are several signal processing techniques known in the art that may be used to determine Δt from the interferograms 408(1) and 408(2) interferograms. One technique is to select the integer values of n₁ and n₂ of the respective bursts 414(1) and 414(2) that are maxima (or minima). However, this approach limits resolution to one-half of a data point, which corresponds to approximately 1 m for many conventional DCS set ups (e.g., d₁ and d₂ on the order of 1 km and f_{c}≈1 Gbps). To improve distance resolution (e.g., to the 1 mm, or less) without increasing f_{c} therefore requires sub-data-point resolution, which can be achieved using interpolation with non-linear regression, Hilbert transforms (i.e., separate amplitude and instantaneous phase signals calculated from each of the interferograms 408(1) and 408(2)), and Fourier transforms, among other techniques. Another approach is to calculate cross-correlation of the interferograms 408(1) and 408(2), which will be maximized at Δt. Thus, cross-correlation may be used to directly determine Δt without having to separately determine t₁ and t₂. Another signal-processing technique for sub-data-point resolution may be used without departing from the scope hereof.

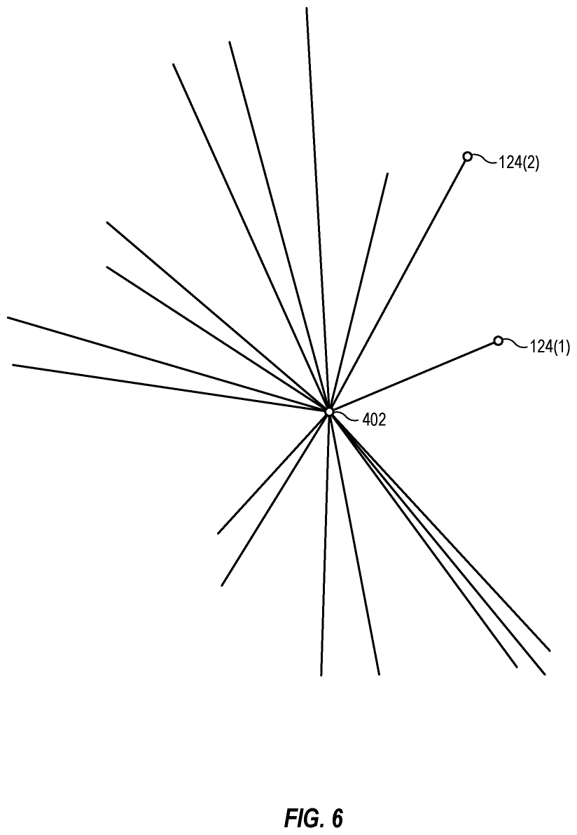

FIG. 6 is a top view of a geographic region in which the optical transceiver 402 can be rotated to work with seventeen retroreflectors 124. Each white line in FIG. 6 denotes the path between the optical transceiver 402 and one of the retroreflectors 124. For clarity in FIG. 6, only two of the retroreflectors 124 are labeled. FIG. 6 illustrates how the measurements shown in FIGS. 4 and 5 can be extended to any number m of retroreflectors 124. Extending the above discussion yields the following set of equations:

$$\begin{matrix}{d}_{1} = {d}^{( 0 )} + α {t}_{1} \\ d = {d}^{( 0 )} + α {t}_{2} \\ ⋮ \\ {d}_{m} = {d}^{( 0 )} + α {t}_{m}\end{matrix} \qquad ( 1 )$$

where α is a constant that depends on the speed of light c and the clock frequency f_{c}. From each measured interferogram 408(i) a corresponding time tᵢ is determined. Altogether, there are m measurements. However, there are m+1 unknowns due to d⁽⁰⁾, which is a common offset that shifts all bursts 414 within the interferograms 408. Thus, for d=0, the resulting burst 414 will not be maximized at the first data point of the resulting interferogram 408.

Due to d⁽⁰⁾, there is always one more unknown than the number of measurements, and therefore Eqn. 1 cannot be solved uniquely. Nevertheless, the measured values t₁, . . . , tₘ provide accurate information that can be used to minimize the number of additional measurements needed to solve Eqn. 1. In some embodiments, only one additional measurement is combined with t₁, . . . , tₘ to uniquely solve for all values d₁, . . . , dₘ. For example, the one additional measurement could be a measurement of distance between the transceiver 102 and one retroreflector 124(i) (i.e., a measurement of dᵢ). The one distance measurement could be performed with a laser rangefinger or LIDAR scanner. Alternatively, the one distance measurement could be performed with a tape measure or another kind of ruler. Alternatively, GPS coordinates of the transceiver 402 and any of the retroreflectors 124(i) could be measured to determine the distance therebetween. Another technique to measure one value of dᵢ may be used without departing from the scope hereof.

Another technique to solve Eqn. 1 is to measure d⁽⁰⁾. For example, a planar mirror could be positioned directly in front of the transceiver 402 such that the distance propagated by the beams 416 and 418 is essentially zero. The resulting interferogram 408 will have a burst 414 centered at a data point that determines d⁽⁰⁾. Alternatively, the retroreflector or planar mirror could be placed a fixed, but known, distance in front of the transceiver 402. In this case, the measured center position of the burst 414 and the known distance can be used to uniquely solve for d⁽⁰⁾.

In other embodiments, more than one additional measurement is used with t₁, . . . , tₘ to solve Eqn. 1. In this case, the number of measurements exceeds the number of unknowns and the values d₁, . . . , dₘ can be obtained by fitting the measured values to a mathematical model. For example, each retroreflector 124 may be tagged with a low-resolution GPS receiver that reports its position. Even though the uncertainty in each GPS position may be several meters, fitting both the GPS positions and the measured values of t₁, . . . , tₘ to a model still yields values of d₁, . . . , dₘ with sufficient accuracy (i.e., less than one meter).

Another approach to solving Eqn. 1 is to use the spectroscopy data obtained by measuring the retroreflected beam 418 to estimate distance. Typically, the absorption signal is divided by the path length to calculate the molecular concentration along the path. However, inversely, the absorption signal could be combined with an estimate of the atmospheric concentration (e.g., a typical global background of a given species, or a humidity sensor if detecting water, etc.) to estimate the path length.

Other techniques to provide the additional constraint needed to solve Eqn. 1 may be used without departing from the scope hereof.

In other embodiments, measured frequency shifts in molecular absorption data obtained with the above DCS systems can be used to calculate the error in f_{c}. The frequency shifts are obtained by comparing the data to a molecular database. These frequency shifts essentially tie α in Eqn. 1 to the molecular database rather than the “assumed” value of f_{c}. Accordingly, this use of the molecular database allows for drift in f_{c} to be determined and correcting, thereby increasing accuracy of the determined values of d₁, . . . , dₘ by reducing the error introduced by α due to uncertainty in f_{c}

### Combination of Features

Features described above as well as those claimed below may be combined in various ways without departing from the scope hereof. The following examples illustrate possible, non-limiting combinations of features and embodiments described above. It should be clear that other changes and modifications may be made to the present embodiments without departing from the spirit and scope of this invention:

(A1) A dual-beam optomechanical steerer includes a two-axis gimbal system and a first rotator that is mounted to the two-axis gimbal system, configured to receive a first optical transmitter, and adjustable to rotate the first optical transmitter. The dual-beam optomechanical steerer also includes a second rotator that is mounted to the two-axis gimbal system, configured to receive a second optical transmitter, and adjustable to rotate the second optical transmitter.

(A2) In the dual-beam optomechanical steerer denoted (A1), the first rotator is adjustable to rotate the first optical transmitter to a first azimuth and a first elevation angle. Furthermore, the second rotator is adjustable to rotate the second optical transmitter to a second azimuth different from the first azimuth, and a second elevation angle different from the first elevation angle.

(A3) In either of the dual-beam optomechanical steerers denoted (A1) and (A2), the first rotator is adjustable to rotate the first optical transmitter over a first azimuthal range and a first elevation angular range. Furthermore, the second rotator is adjustable to rotate the second optical transmitter over a second azimuthal range and a second elevation angular range.

(A4) In the dual-beam optomechanical steerer denoted (A3), the first and second azimuthal ranges are similar and the first and second elevation angular ranges are similar.

(A5) In any of the dual-beam optomechanical steerers denoted (A1) to (A4), the two-axis gimbal system includes a motor.

(A6) In any of the dual-beam optomechanical steerers denoted (A1) to (A5), each of the first and second rotators includes a motor.

(A7) In any of the dual-beam optomechanical steerers denoted (A1) to (A6), each of the first and second rotators includes a piezoelectric actuator.

(A8) In any of the dual-beam optomechanical steerers denoted (A1) to (A7), each of the first and second rotators is one of a tip-tilt stage, a ball-and-socket stage, a two-axis goniometer, and a one-axis goniometer combined with a rotation stage.

(A9) In any of the dual-beam optomechanical steerers denoted (A1) to (A8), each of the first and second rotators includes a pair of rotatable wedges.

(A10) In the dual-beam optomechanical steerer denoted (A9), each of the first and second rotators further includes a pair of rotation stages for rotating the pair of rotatable wedges.

(A11) In any of the dual-beam optomechanical steerers denoted (A1) to (A10), the dual-beam optomechanical steerer further includes the first and second optical transmitters.

(A12) In any of the dual-beam optomechanical steerers denoted (A1) to (A11), each of the first and second optical transmitters is an optical transceiver.

(B1) A beamsteering method includes adjusting a two-axis gimbal system, adjusting a first rotator to rotate a first optical transmitter, and adjusting a second rotator to rotate a second optical transmitter. The first and second rotators are mounted to the two-axis gimbal system.

(B2) In the beamsteering method denoted (B1), said adjusting the first rotator includes rotating the first optical transmitter to a first azimuth and a first elevation angle. Furthermore, said adjusting the second rotator includes rotating the second optical transmitter to a second azimuth different from the first azimuth, and a second elevation angle different from the first elevation angle.

(B3) In either of the beamsteering methods denoted (B1) and (B2), said adjusting the first rotator includes controlling at least one motor of the first rotator to deviate the first optical transmitter. Furthermore, said adjusting the second rotator includes controlling at least one motor of the second rotator to deviate the second optical transmitter.

(B4) In any of the beamsteering methods denoted (B1) to (B3), said adjusting the first rotator includes controlling at least one piezoelectric actuator of the first rotator to rotate the first optical transmitter. Furthermore, said adjusting the second rotator includes controlling at least one piezoelectric actuator of the second rotator to rotate the second optical transmitter.

(B5) In any of the beamsteering methods denoted (B1) to (B4), said adjusting the first rotator includes actuating one of a first tip-tilt stage, a first ball-and-socket stage, a first two-axis goniometer, and a first one-axis goniometer combined with a first rotation stage. Said adjusting the second rotator includes actuating one of a second tip-tilt stage, a second ball-and-socket stage, a second two-axis goniometer, and a second one-axis goniometer combined with a second rotation stage.

(B6) In any of the beamsteering methods denoted (B1) to (B5), said adjusting the first rotator includes rotating a first pair of wedges. Said adjusting the second rotator includes rotating a second pair of wedges.

(B7) In any of the beamsteering methods denoted (B1) to (B6), the beamsteering method further includes transmitting a first optical beam with the first optical transmitter and transmitting a second optical beam with the second optical transmitter.

(B8) In any of the beamsteering methods denoted (B1) to (B7), the beamsteering method further includes mounting the first optical transmitter to the first rotator and mounting the second optical transmitter to the second rotator.

(B9) In any of the beamsteering methods denoted (B1) to (B8), the first optical transmitter is a first optical transceiver and the second optical transmitter is a second optical transceiver. The beamsteering method further includes simultaneously receiving a first laser beam with the first optical transceiver and receiving a second laser beam with the second optical transceiver.

(C1) A dual-beam optomechanical steerer includes a two-axis gimbal system and a first optical deflector mounted to the two-axis gimbal system. The first optical deflector is adjustable to deflect a first optical beam transmitted by a first optical transmitter mounted to the two-axis gimbal system. The dual-beam optomechanical steerer also includes a second optical deflector mounted to the two-axis gimbal system. The second optical deflector is adjustable to deflect a second optical beam transmitted by a second optical transmitter mounted to the two-axis gimbal system.

(C2) In the dual-beam optomechanical steerer denoted (C1), the first optical deflector is adjustable to deflect the first optical beam to a first azimuth and a first elevation angle. The second optical deflector is adjustable to deflect the second optical beam to a second azimuth different from the first azimuth, and a second elevation angle different from the first elevation angle.

(C3) In either of the dual-beam optomechanical steerers denoted (C1) and (C2), the first optical deflector is adjustable to deflect the first optical beam over a first azimuthal range and a first elevation angular range. The second optical deflector is adjustable to deflect the second optical beam over a second azimuthal range and a second elevation angular range.

(C4) In the dual-beam optomechanical steerer denoted (C3), the first and second azimuthal ranges are similar and the first and second elevation angular ranges are similar.

(C5) In any of the dual-beam optomechanical steerers denoted (C1) to (C4), the two-axis gimbal system includes a motor.

(C6) In any of the dual-beam optomechanical steerers denoted (C1) to (C5), each of the first and second optical deflectors includes a motor.

(C7) In any of the dual-beam optomechanical steerers denoted (C1) to (C6), each of the first and second optical deflectors includes a piezoelectric actuator.

(C8) In any of the dual-beam optomechanical steerers denoted (C1) to (C7), each of the first and second optical deflectors is one of a translatable lens, a rotatable pair of refractive optical elements, a rotatable pair of diffractive optical elements, an acousto-optic deflector, and a mirror mount.

(C9) In the dual-beam optomechanical steerer denoted (C8), the rotatable pair of refractive optical elements includes a Risley prism.

(C10) In the dual-beam optomechanical steerer denoted (C9), the dual-beam optomechanical steerer includes a pair of rotation stages for rotating the Risley prism.

(C11) In any of the dual-beam optomechanical steerers denoted (C1) to (C10), the dual-beam optomechanical steerer further includes the first and second optical transmitters.

(C12) In any of the dual-beam optomechanical steerers denoted (C1) to (C11), each of the first and second optical transmitters is an optical transceiver.

(D1) A beamsteering method includes adjusting a two-axis gimbal system, adjusting a first optical deflector to deflect a first optical beam transmitted by a first optical transmitter mounted to the two-axis gimbal system, and adjusting a second optical deflector to deflect a second optical beam transmitted by a second optical transmitter mounted to the two-axis gimbal system.

(D2) In the beamsteering method denoted (D1), said adjusting the first optical deflector includes deflecting the first optical beam to a first azimuth and a first elevation angle. Said adjusting the second optical deflector includes deflecting the second optical beam to a second azimuth different from the first azimuth, and a second elevation angle different from the first elevation angle.

(D3) In either of the beamsteering methods denoted (D1) and (D2), said adjusting the first optical deflector includes controlling at least one motor of the first optical deflector. Said adjusting the second optical deflector includes controlling at least one motor of the second optical deflector.

(D4) In any one of the beamsteering methods denoted (D1) to (D3), said adjusting the first optical deflector includes controlling at least one piezoelectric actuator of the first optical deflector. Said adjusting the second optical deflector includes controlling at least one piezoelectric actuator of the second optical deflector.

(D5) In any one of the beamsteering methods denoted (D1) to (D4), said adjusting the first optical deflector includes translating a first lens through which the first optical beam propagates. Said adjusting the second optical deflector includes translating a second lens through which the second optical beam propagates.

(D6) In any one of the beamsteering methods denoted (D1) to (D5), said adjusting the first optical deflector includes rotating a first Risley prism pair through which the first optical beam propagates. Said adjusting the second optical deflector includes rotating a second Risley prism pair through which the second optical beam propagates.

(D7) In any one of the beamsteering methods denoted (D1) to (D6), the beamsteering method further includes mounting the first and second optical transmitters to the two-axis gimbal system.

(D8) In any one of the beamsteering methods denoted (D1) to (D7), the first optical transmitter is a first optical transceiver the second optical transmitter is a second optical transceiver. The beamsteering method further includes simultaneously (i) receiving, with the first optical transceiver, a third optical beam that propagates through the first optical deflector and (ii) receiving, with the second optical transceiver, a fourth incoming optical beam that propagates through the second optical deflector.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

1. A dual-beam optomechanical steerer, comprising:
a two-axis gimbal system;
a first rotator mounted to the two-axis gimbal system, configured to receive a first optical transmitter, and adjustable to rotate the first optical transmitter; and
a second rotator mounted to the two-axis gimbal system, configured to receive a second optical transmitter, and adjustable to rotate the second optical transmitter.

2. The dual-beam optomechanical steerer of claim 1, wherein:
the first rotator is adjustable to rotate the first optical transmitter to a first azimuth and a first elevation angle; and
the second rotator is adjustable to rotate the second optical transmitter to a second azimuth different from the first azimuth, and a second elevation angle different from the first elevation angle.

3. The dual-beam optomechanical steerer of claim 1, wherein:
the first rotator is adjustable to rotate the first optical transmitter over a first azimuthal range and a first elevation angular range; and
the second rotator is adjustable to rotate the second optical transmitter over a second azimuthal range and a second elevation angular range.

4. The dual-beam optomechanical steerer of claim 3, wherein:
the first and second azimuthal ranges are similar; and
the first and second elevation angular ranges are similar.

5. The dual-beam optomechanical steerer of claim 1, the two-axis gimbal system including a motor.

6. The dual-beam optomechanical steerer of claim 1, each of the first and second rotators including a motor.

7. The dual-beam optomechanical steerer of claim 1, each of the first and second rotators including a piezoelectric actuator.

8. The dual-beam optomechanical steerer of claim 1, wherein each of the first and second rotators is one of a tip-tilt stage, a ball-and-socket stage, a two-axis goniometer, and a one-axis goniometer combined with a rotation stage.

9. The dual-beam optomechanical steerer of claim 1, each of the first and second rotators comprising a pair of wedges.

10. The dual-beam optomechanical steerer of claim 9, each of the first and second rotators further comprising a pair of rotation stages for rotating the pair of wedges.

11. The dual-beam optomechanical steerer of claim 1, further comprising the first and second optical transmitters.

12. The dual-beam optomechanical steerer of claim 1, wherein each of the first and second optical transmitters is an optical transceiver.

13. A beamsteering method, comprising:
adjusting a two-axis gimbal system;
adjusting a first rotator to rotate a first optical transmitter; and
adjusting a second rotator to rotate a second optical transmitter;
wherein the first and second rotators are mounted to the two-axis gimbal system.

14. The beamsteering method of claim 13, wherein:
said adjusting the first rotator includes rotating the first optical transmitter to a first azimuth and a first elevation angle; and
said adjusting the second rotator includes rotating the second optical transmitter to a second azimuth different from the first azimuth, and a second elevation angle different from the first elevation angle.

15. The beamsteering method of claim 13, wherein:
said adjusting the first rotator includes controlling at least one motor of the first rotator to deviate the first optical transmitter; and
said adjusting the second rotator includes controlling at least one motor of the second rotator to deviate the second optical transmitter.

16. The beamsteering method of claim 13, wherein:
said adjusting the first rotator includes controlling at least one piezoelectric actuator of the first rotator to rotate the first optical transmitter; and
said adjusting the second rotator includes controlling at least one piezoelectric actuator of the second rotator to rotate the second optical transmitter.

17. The beamsteering method of claim 13, wherein:
said adjusting the first rotator includes actuating one of a first tip-tilt stage, a first ball-and-socket stage, a first two-axis goniometer, and a first one-axis goniometer combined with a first rotation stage; and
said adjusting the second rotator includes actuating one of a second tip-tilt stage, a second ball-and-socket stage, a second two-axis goniometer, and a second one-axis goniometer combined with a second rotation stage.

18. The beamsteering method of claim 13, wherein:
said adjusting the first rotator includes rotating a first pair of wedges; and
said adjusting the second rotator includes rotating a second pair of wedges.

19. The beamsteering method of claim 13, further comprising simultaneously:
transmitting a first optical beam with the first optical transmitter; and
transmitting a second optical beam with the second optical transmitter.

20. The beamsteering method of claim 13, further comprising:
mounting the first optical transmitter to the first rotator; and
mounting the second optical transmitter to the second rotator.

21. The beamsteering method of claim 13, wherein:
the first optical transmitter is a first optical transceiver;
the second optical transmitter is a second optical transceiver; and
the beamsteering method further includes simultaneously:
receiving a first laser beam with the first optical transceiver; and
receiving a second laser beam with the second optical transceiver.

22-42. (canceled)
